(19) United States
(12) Patent Application Publication
(10) Pub. No.: US 2004/0242973 A1
(43) Pub. Date: Dec. 2, 2004

(54) Health care system, health care apparatus, health care method and health care program

(75) Inventors: Yoshinori Tanabe, Osaka (JP); Hirokazu Genno, Osaka (JP)

Correspondence Address: McDERMOTT, WILL & EMERY, 600 13th Street, N.W., Washington, DC, 20005-3096, US

(73) Assignee: SANYO ELECTRIC CO., LTD

(21) Appl. No.: 10/855,335

(22) Filed: May 28, 2004

(30) Foreign Application Priority Data
May 29, 2003 (JP) 2003-152237

(51) Int. Cl. A61B 5/00

(52) U.S. Cl. 600/300; 128/903; 128/904; 128/920

(57) ABSTRACT

A medical question presenting module regularly presents a medical question on a user terminal. An advice selection module selects any of a plurality of advice algorithms recorded in an algorithm database based on physiological amount data, an answer to the medical question, and a personal set value, and presents on the user terminal the selected advice algorithm as an automatic advice, while requesting a doctor terminal for a judgment when the automatic advice cannot be presented. The doctor terminal presents an individual advice on the user terminal via a personal board in an individual advice module. An advice construction module extracts a Q&A history from a Q&A history database, to generate an advice algorithm.

### BACKGROUND OF THE INVENTION

1. Field of the Invention

The present invention relates to a health care system, a health care apparatus, a health care method, and a health care program.

2. Description of the Background Art

A system has been proposed which records daily physiological amount data of a patient via a network, and provides advice automatically (refer to JP 2000-514938 A, for example). The physiological amount data here denotes data relating to life which can be measured objectively, such as body temperature, blood pressure, blood glucose level, and body weight.

In the conventional system, however, it is difficult to judge automatically each condition of a patient to provide advice.

In addition, the above conventional system has allowed informing a doctor of diagnostic results. The doctor is, however, required to confirm all the supplied diagnostic results, resulting in an increase of his/her workload.

Further, formation of advice provided by this system consists of extracting the know-how of a professional such as a doctor, and accordingly requires much effort.

### SUMMARY OF THE INVENTION

An object of the present invention is to provide a health care system, a health care apparatus, a health care method, and a health care program which are capable of efficiently presenting appropriate advice according to the condition of a user.

A health care system according to one aspect of the present invention comprises: a user terminal which is used by a user; a professional terminal which is used by a professional; and a host computer, wherein the user terminal, the professional terminal, and the host computer are interconnected via a communication line, and the host computer includes: a data storage device that stores data relating to the health condition of the user supplied by the user terminal; an advice storage device that stores a plurality of advices relating to the health condition as first advices; a first advice presenting unit that determines whether or not a first advice can be presented based on the data stored in the data storage device, and when a first advice can be presented, selects any of the plurality of first advices stored in the advice storage device to present the advice on the user terminal, while requesting the professional terminal for a judgment when the first advice cannot be presented; and a second advice presenting unit that presents on the user terminal advice from the professional terminal as a second advice.

In the health care system, the user terminal used by the user, the professional terminal used by the professional, and the host computer, are interconnected via the communication line.

In the host computer, the data on the health condition of the user supplied by the user terminal is stored in the data storage device, and the plurality of advices relating to the health condition are stored as the first advices in the advice storage device. The first advice presenting unit determines whether or not the first advice can be presented based on the data stored in the data storage device, and when the first advice can be presented, any of the plurality of first advices stored in the advice storage device is selected to be presented on the user terminal, while when the first advice cannot be presented, the professional terminal is requested for a judgment. Then, the advice from the professional terminal is presented by the second advice presenting unit on the user terminal as the second advice.

In this way, at normal times the first advice is automatically presented to the user based on the data relating to the health condition. This allows the user to readily receive advice. Further, when the first advice cannot be presented, the second advice according to the judgment of the professional is presented to the user. Therefore, when it is difficult to make a judgment, the user can readily receive appropriate advice from the professional. In this case, since the professional is requested for a judgment when the first advice cannot be presented, the workload for the professional does not increase.

Consequently, it is possible to efficiently present appropriate advice according to the condition of the user.

The host computer may construct a first advice based on the second advice presented on the user terminal by the second advice presenting unit, and may further comprise an advice construction unit that makes the advice storage device store the constructed first advice.

In this case, the first advice is constructed by the advice construction unit based on the second advice presented on the user terminal by the second advice presenting unit, and the constructed first advice is stored in the advice storage device. In this way, the first advice is automatically constructed from the professional advice. Consequently, the function for presenting the first advice is enhanced in performance without requiring much effort.

The second advice presenting unit may include an information storage device which can be written to and read from by the user terminal and the professional terminal, and the advice construction unit may construct the first advice based on the information written to the information storage device.

In this case, the information is written to and read from the information storage device by the user terminal and the professional terminal. Then, the advice construction unit constructs the first advice based on the information written to the information storage device. This allows acquisition of professional know-how without requiring much effort, leading to further enhanced performance of the function of presenting the first advice.

The host computer may further comprise a criterion value storage device that stores a criterion value set for each user for presenting advice, and the first advice presenting unit may determine if a first advice can be presented and select a first advice, based on the data stored in the data storage device and the criterion value stored in the criterion value storage device.

In this case, the criterion value set for each user is stored in the criterion value storage device, and the first advice presenting unit determines whether or not the first advice can be presented and selects the first advice, based on the data stored in the data storage device and the criterion value stored in the criterion value storage device. This allows presenting advice according to the health condition of the user.

The criterion value stored in the criterion value storage device may be set and changed by the professional terminal.

In this case, since the professional can set or change the criterion value by the professional terminal, advice can be presented based on more appropriate criteria values.

When a first advice cannot be presented, the first advice presenting unit may determine if there is a serious problem based on the criterion value stored in the criterion value storage device.

In this case, when the first advice cannot be presented, the first advice presenting unit determines whether or not there is a serious problem, so that an appropriate approach can be taken according to the degree of the health condition of the user.

The professional terminal may include a plurality of terminals having a hierarchical structure, and when a first advice cannot be presented, the first advice presenting unit may request a judgment to a highest level terminal of the plurality of terminals having the hierarchical structure.

In this case, the plurality of terminals have the hierarchical structure, and the highest level terminal is requested for a judgment when the first advice cannot be presented, so that the request for a judgment can be readily made.

The professional terminal may include a plurality of terminals, and when a first advice cannot be presented, the first advice presenting unit may select any of the plurality of terminals and request the selected terminal for a judgment, based on the data stored in the data storage device.

In this case, since an appropriate professional terminal is requested for a judgment based on the data, more appropriate advice can be quickly presented.

The host computer may further comprise a medical question presenting unit that presents a medical question on the user terminal, the data storage device may store as the data user's physiological amount data supplied by the user terminal and an answer to the medical question supplied by the user terminal in response to the medical question by the medical question presenting unit, and the first advice presenting unit may determine if a first advice can be presented and selects a first advice, based on the physiological amount data and the answer stored in the data storage device.

In this case, the medical question is presented on the user terminal by the medical question presenting unit, and the user's physiological amount data and answer to the medical question which are supplied by the user terminal are stored as the data by the data storage device. Then, based on the physiological amount data and the answer, it is determined if the first advice can be presented and the selection of the first advice is made. This allows the first advice compatible with the health condition of the user to be presented.

When the data falls short to determine if the first advice can be presented and select the first advice, the first advice presenting unit may acquire sufficient data from the medical question performed by the medical question presenting unit.

In this case, when the data falls short to determine if the first advice can be presented and select the first advice, sufficient data is acquired from the medical question performed by the medical question presenting unit. This allows accurate selection of the first advice.

A health care apparatus according to another aspect of the present invention comprises: a data storage device that stores data relating to the health condition of a user supplied by the user; an advice storage device that stores a plurality of advices relating to the health condition as first advices; a first advice presenting unit that determines whether or not a first advice can be presented based on the data stored in said data storage device, and when a first advice can be presented, selects any of the plurality of first advices stored in the advice storage device to present the advice to the user, while requesting a professional for a judgment when the first advice can be presented; and a second advice presenting unit that presents advice from the professional to the user as a second advice.

In the health care apparatus, the data relating to the health condition of the user supplied by the user is stored in the data storage device, and the plurality of advices relating to the health condition are stored as the first advices in the advice storage device. It is determined by the first advice presenting unit if the first advice can be presented based on the data stored in the data storage device, and when the first advice can be presented, any of the plurality of first advices stored in the advice storage device is selected to be presented to the user, while the professional is requested for a judgment when the first advice cannot be presented. Then, the advice from the professional is presented by the second advice presenting unit to the user as the second advice.

In this way, at normal times, the first advice is automatically presented to the user based on the data relating to the health condition. Therefore, the user can readily receive advice. When the first advice cannot be presented, the second advice according to the judgment of the professional is presented to the user. Therefore, when it is difficult to make a judgment, the user can readily receive appropriate advice from the professional. In this case, since the professional is requested for a judgment when the first advice cannot be presented, his/her workload does not increase.

It is therefore possible to efficiently present appropriate advice according to the condition of the user.

The health care apparatus may further comprise an advice construction unit that constructs a first advice based on the second advice presented to the user by the second advice presenting unit, and makes the advice storage device store the constructed first advice.

In this case, the first advice is constructed by the advice construction unit based on the second advice presented to the user by the second advice presenting unit, and the constructed first advice is stored in the advice storage device. In this way, the first advice is automatically constructed from the professional advice. This allows enhanced performance of the function of presenting the first advice without requiring much effort.

The second advice presenting unit may include an information storage device which can be written to and read from by the user and the professional, and the advice construction unit may construct the first advice based on the information written to the information storage device.

In this case, the information is written to and read from the information storage device by the user and the professional. Then, the first advice is constructed by the advice construction unit, based on the information written to the information storage device. It is therefore possible to acquire professional know-how without requiring much effort, leading to further enhanced the performance of the function of presenting the first advice.

The health care apparatus may further comprise a medical question presenting unit that presents a medical question to the user, wherein the data storage device stores as said data user's physiological amount data supplied by the user and an answer supplied by the user in response to the medical question by the medical question presenting unit, and the first advice presenting unit determines whether or not a first advice can be presented and selects the first advice, based on the physiological amount data and the answer stored in the data storage device.

In this case, the medical question is presented to the user by the medical presenting unit, and the physiological amount data and answer to the medical question which are supplied by the user are stored as the data by the data storage device. Determination is then made if the first advice can be presented and selection of the first advice is made, based on the physiological amount data and answer. This allows the first advice which is more compatible with the health condition of the user to be presented.

When the data falls short to determine if the first advice can be presented and select the first advice, the first advice presenting unit may acquire sufficient data from the medical question performed by the medical question presenting unit.

In this case, when the data falls short to determine if the first advice can be presented and to select the first advice, sufficient data is acquired from the medical question performed by the medical presenting unit. This allows accurate selection of the first advice.

A health care method according to still another aspect of the present invention may comprise the steps of: storing data relating to the health condition of a user supplied by the user; storing a plurality of advices relating to the health condition as first advices; determining if a first advice can be presented based on the stored data, and when a first advice can be presented, selecting any of the stored plurality of first advices to present the advice to the user, while requesting a professional for a judgment when the first advice cannot be presented; and presenting advice from the professional to the user as a second advice.

In the health care method, the data relating to the health condition of the user supplied by the user is stored, and the plurality of advices relating to the health condition are stored as the first advices. It is determined if the first advice can be presented based on the stored data, and when the first advice can be presented, any of the stored plurality of first advices is selected to be presented to the user, while the professional is requested for a judgment when the first advice cannot be presented. The advice from the professional is then presented to the user as the second advice.

In this way, at normal times, the first advice is automatically presented to the user based on the data relating to the health condition. The user can therefore readily receive advice. When the first advice cannot be presented, the second advice according to the judgment by the professional is presented to the user. Therefore, when it is difficult to make a judgment, the user can readily receive appropriate advice from the professional. In this case, since the professional is requested for a judgment when the first advice cannot be presented, the workload of the professional does not increase.

It is therefore possible to efficiently present appropriate advice according to the condition of the user.

The health care method may further comprise the step of constructing a first advice based on the second advice presented to the user, and storing the constructed first advice.

In this case, the first advice is constructed based on the second advice presented to the user, and the constructed first advice is stored. In such a way, the first advice is automatically constructed from the professional advice. This allows enhanced performance of the function of presenting the first advice without requiring much effort.

The step of constructing advice may include constructing the first advice based on information stored in an information storage device which can be written to and read from by the user and the professional.

In this case, the information is written to and read from the information storage device by the user and the professional. The first advice is then constructed based on the information written to the information storage device. Therefore, it is possible to acquire professional know-how, leading to further enhanced performance of the function of presenting the first advice.

The health care method may further comprise the step of presenting a medical question to the user, the step of storing data including storing as the data user's physiological amount data supplied by the user and an answer supplied by the user in response to the medical question, and the step of presenting the first advice including determining if a first advice can be presented and selecting the first advice, based on the stored physiological amount data and answer.

In this case, the user is presented with the medical question, and the physiological amount data and the answer to the medical question which are supplied by the user are stored as the data. Then, determination is made if the first advice can be presented and selection of the first advice is made, based on the physiological amount data and answer. This allows the first advice which is more compatible with the health condition of the user to be presented.

The step of presenting the first advice may include acquiring sufficient data from the medical question, when data falls short to determine if the first advice can be presented and select the first advice.

In this case, when the data falls short to determine if the first advice can be presented and to select the first advice, sufficient data is acquired from the medical question. This allows accurate selection of the first advice.

A health care program according to still another aspect of the present invention is a computer-readable health care program which allows the computer to implement the processes of: storing data relating to the health condition of a user supplied by the user; storing a plurality of advices relating to the health condition as first advices; determining if a first advice can be presented based on the stored data, and when a first advice can be presented, selecting any of the stored plurality of first advices to present the advice to the user, while requesting a professional for a judgment when the first advice cannot be presented; and presenting advice from the professional as a second advice to the user.

According to the health care program, the data relating to the condition of the user supplied by the user is stored, and the plurality of advices relating to the health condition are stored as the first advices. It is determined if the first advice can be presented based on the stored data, and when the first advice can be presented, any of the stored plurality of first advices is selected to be presented to the user, while the professional is requested for a judgment when the first advice cannot be presented. Then, the advice from the professional is presented to the user as the second advice.

In this way, at normal times, the first advice is presented automatically to the user based on the data relating to the health condition. Therefore, the user can readily receive advice. When the first advice cannot be presented, the second advice according to the judgment by the professional is presented to the user. When it is difficult to make a judgment, therefore, the user can readily receive appropriate advice from the professional. In this case, since the professional is requested for a judgment when the first advice cannot be presented, the workload of the professional does not increase.

Consequently, it is possible to efficiently present appropriate advice according to the condition of the user.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a schematic view showing the structure of a heath care system according to an embodiment of the present invention;

FIG. 2 is a block diagram showing the structure of a server of the health care system of FIG. 1;

FIG. 3 is a block diagram showing the structure and operation of a health care program;

FIG. 4 is a block diagram showing the structure and operation of the health care program;

FIG. 5 is a flowchart showing the operation of the advice selection module of FIG. 3 and FIG. 4;

FIG. 6 is a flowchart showing the task of a doctor that uses a doctor terminal;

FIG. 7 is a flowchart showing the operation of the individual advice module of FIG. 3 and FIG. 4;

FIG. 8 is a flowchart showing the operation of the advice construction module of FIG. 3 and FIG. 4;

FIG. 9 is a flowchart showing a process of recording an algorithm in the operation of the advice construction module of FIG. 3 and FIG. 4.

### DESCRIPTION OF THE PREFERRED EMBODIMENTS

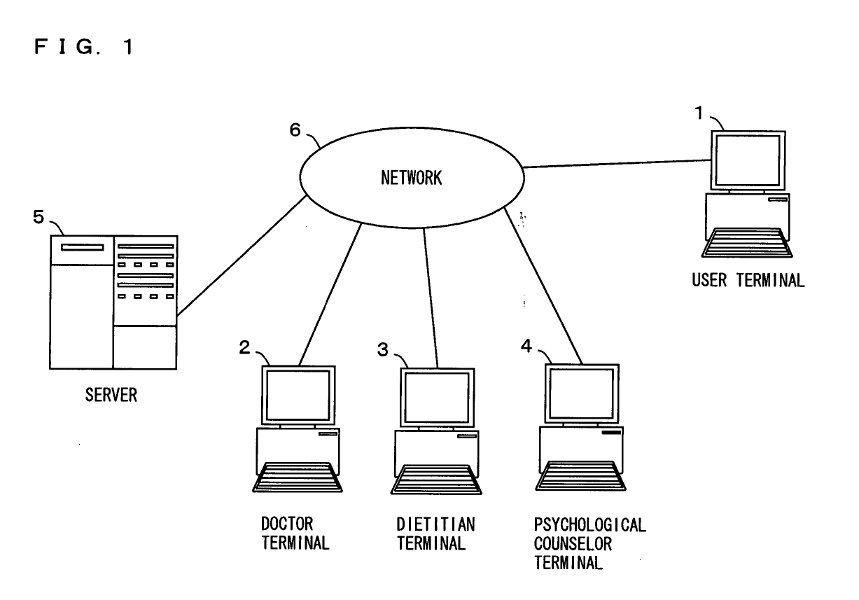

FIG. 1 is a schematic view showing the structure of a health care system according to an embodiment of the present invention. The health care system of FIG. 1 includes a user terminal 1, a doctor terminal 2, a dietitian terminal 3, a psychological counselor terminal 4, and a server 5. The user terminal 1, doctor terminal 2, dietitian terminal 3, psychological counselor terminal 4, and server 5 are interconnected via a network 6 which is the Internet, a public network or the like.

The user terminal 1 is composed of a personal computer or the like, and used by a user. The user terminal 1 has functions of recording physiological amount data, basic information (name, sex, date of birth, and the like), dietary information (contents of meals, images of meals, calorie intake), examination results, exercise amount information, and a function of answering medical questions. The physiological amount data denotes data relating to life which can be measured objectively, such as blood pressure, blood glucose level, and body weight, and is also termed vital data.

The doctor terminal 2, dietitian terminal 3, and psychological counselor terminal 4 are each composed of personal computers and the like, and they are terminal equipment used by a doctor, a dietitian, and a psychological counselor, respectively. Here, the doctor, dietitian, and psychological counselor are generically termed professionals. Although in the health care system of this embodiment, the doctor, dietitian, and psychological counselor are adopted as professionals, any other professionals may be adopted in the health care system. The terminal used by a professional may have the similar recording functions as the user terminal 1. The doctor terminal 2 may have the function of recording examination results, and the dietitian terminal ˜3 may have the function of recording dietary information.

The server 5 is a host computer. The server 5 operates according to the health care program described below, manages data for implementing the health care system, and has functions of presenting an automatic advice and presenting an individual advice.

Here, the automatic advice denotes advice which is presented automatically based on the physiological amount data, answers to medical questions, and individual set values. The automatic advice is presented upon recording the user supplied physiological amount data or answers to medical questions or upon request for advice from the user.

The individual advice denotes advice which is presented based on the judgment of a professional when it is not possible to present the automatic advice. The individual set value denotes a criterion value for judging if the user has a serious health problem, which is set by the professional for each user. Examples of the individual set values include criteria values for body weight, body pressure, blood glucose level, and the like.

Note that when an individual advice is to be presented or when the professional has not yet judged the necessity for individual advice, annotation is supplied to the automatic advice.

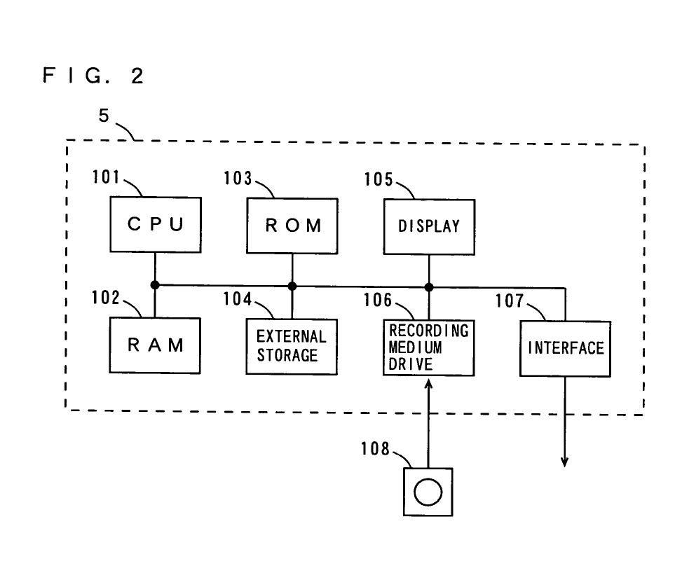

FIG. 2 is a block diagram showing the structure of the server 5 of the health care system of FIG. 1.

As shown in FIG. 2, the server 5 includes a CPU (Central Processing Unit) 101, a RAM (Random Access Memory) 102, a ROM (Read Only Memory) 103, an external storage device 104, a display 105, a recording medium drive 106, and an interface 107.

The interface 107 is connected to the network 6. The ROM 103 stores a system program. The recording medium drive 106 is composed of a CD-ROM (Compact Disc-Read Only Memory) drive, a floppy disc drive, or the like which reads and writes data from and to a recording medium 108, which is a CD-ROM, a floppy disc, or the like. In the recording medium 108, a health care program is recorded.

The external storage device 104 is composed of a hard disc or the like, and stores the health care program read from the recording medium 108 via the recording medium drive 106. The CPU 101 executes on the RAM 102 the health care program stored in the external storage device 104. The display 105 is composed of a CRT (Cathode Ray Tube), a liquid crystal display, or the like, displaying a variety of information.

Note that as the recording medium 108 for recording the health care program, any of various types of storage mediums may be used, such as a semiconductor memory, for example, a ROM, a hard disc, and the like. Further, the health care program may be downloaded via the network 6 into the external storage device 104 to be executed on the RAM 102.

Note that the structures of the user terminal 1, doctor terminal 2, dietitian terminal 3, and psychological counselor terminal 4 in the health care system of FIG. 1 are the same as that of the server 5 of FIG. 2.

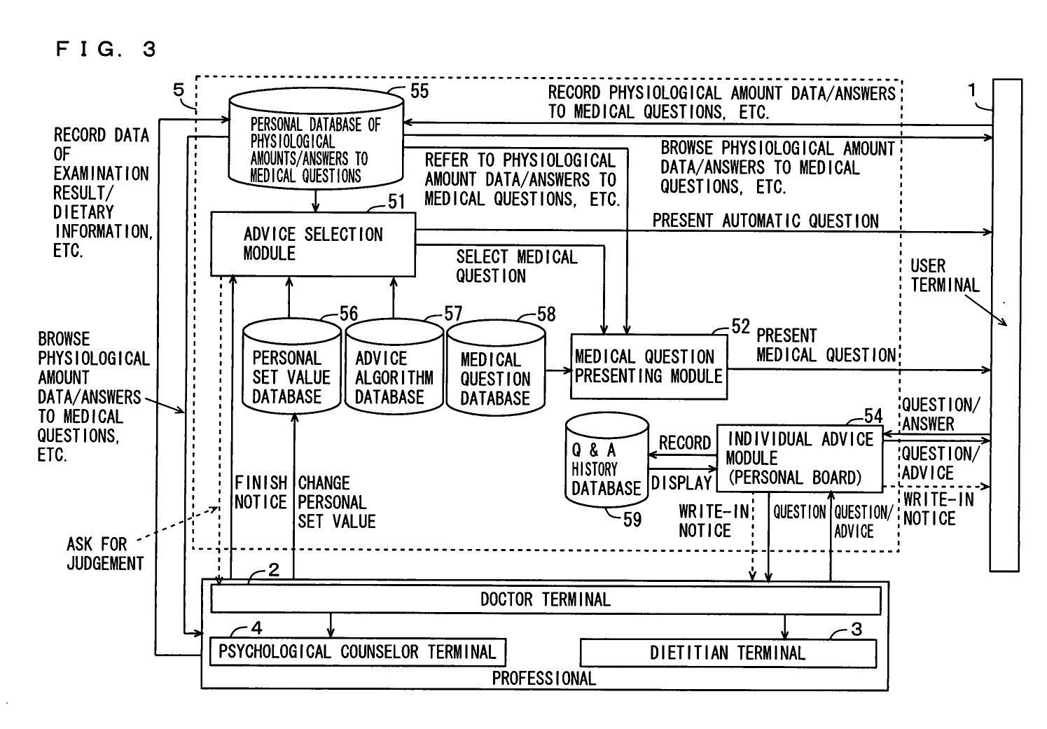

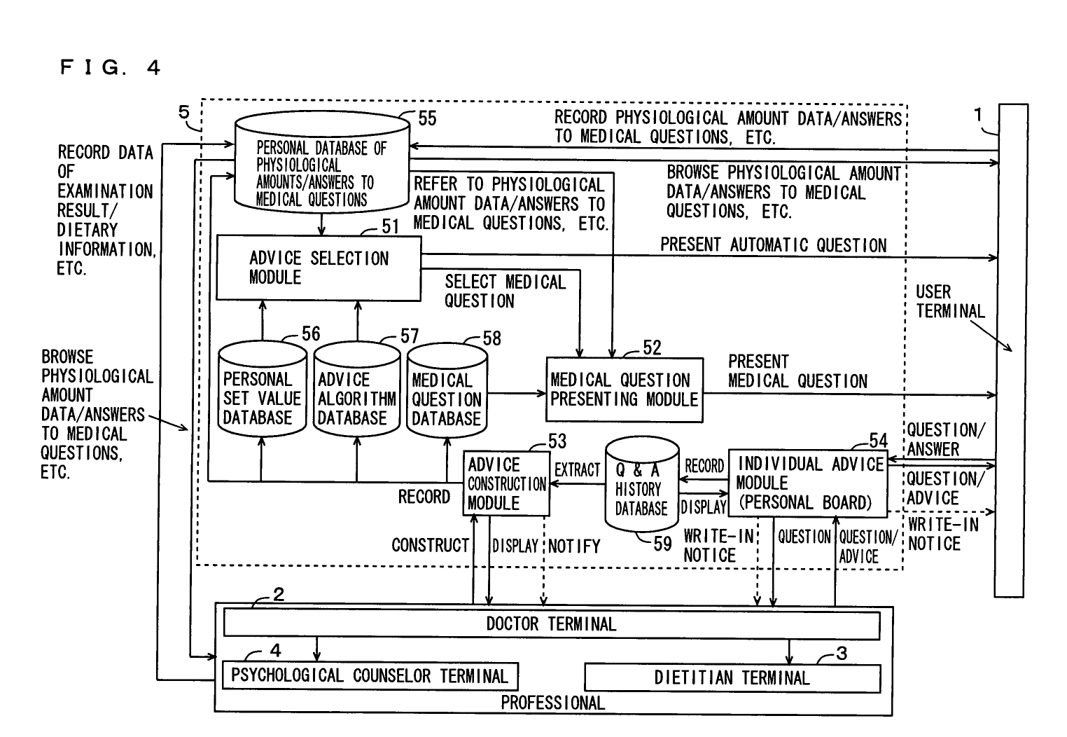

FIGS. 3 and 4 are block diagrams showing the structure and operation of the health care program. FIG. 3 shows switching between an automatic advice and an individual advice, and FIG. 4 shows generation of an automatic advice from an individual advice.

As shown in FIGS. 3 and 4, the health care program includes an advice selection module 51, a medical question presenting module 52, an advice construction module 53, an individual advice module 54, a personal database of physiological amounts/answers to medical questions (hereinafter abbreviated to a personal database) 55, a personal set value database 56, an advice algorithm database (hereinafter abbreviated as an algorithm database) 57, a medical question database 58, and a Q&A history database 59.

The personal advice module 54 has an electronic bulletin board (hereinafter referred to as a personal board) for each individual. When the professional writes into the personal board, a write-in notice is transmitted to the user by e-mail, and when the user writes into the personal board, a write-in notice is transmitted to the professional by e-mail. This write-in notice only recites how to access the personal board while reciting no contents of the written messages. This maintains the secrecy of the contents of responses between the user and the professional.

Further, the history of unread and read messages on the personal board is recorded for each user and professional.

The personal database 55 stores the physiological amount data, dietary information, examination results, an amount of exercise, and answers to medical questions for each individual which are recorded by the user terminal 1. The individual database 55 also stores the dietary information, examination results, and the like which are recorded by the doctor terminal 2 or dietitian terminal 3. The information stored in the personal data 55 will hereinafter be refereed to as personal data. Personal set values for each individual are recorded in the personal set value database 56.

A plurality of advice algorithms are recorded in the algorithm database 57. Here, the advice algorithm denotes a procedure for an automatic advice.

A plurality of medical question sentences are recorded in the medical question database 58. In the Q&A history database 59, the history of questions and answers between the user and doctor (hereinafter referred to as a Q&A history) is recorded.

Referring to FIG. 3, description will first be made of switching between the automatic advice and the individual advice.

The medical question presenting module 52 regularly receives an instruction for selecting a medical question from the advice selection module 5I, selects a medical question sentence recorded in the medical question database 58, and presents the medical question on the user terminal 1. The medical question presenting module 52 also refers to the personal data recorded in the personal database, and in the case where the personal data falls short, it selects a medical question sentence recorded in the medical question database 58 to present the medical question on the user terminal 1. The medical question may be performed when the amount of the personal data proves to be short, or that it is a preferential medical question may be stored in the medical question presenting module 52 for the medical question to be performed next time.

The user answers the medical question presented on the user terminal 1, and records his/her personal data in the personal database 55. The user can also browse the personal data recorded in the personal database 55 using the user terminal 1.

The advice selection module 51 selects any of the plurality of advice algorithms recorded in the algorithm database 57 based on the personal data recorded in the personal database 55 and the personal set values recorded in the personal set value database 56, to present on the user terminal 1 the selected algorithm as an automatic advice. In the case where it is not possible to present an automatic advice, the advice selection module 51 transmits only the objective facts to the user terminal 1, and requests the doctor terminal 2 for a judgment by e-mail.

The doctor browses the personal data recorded in the personal database 55 using the doctor terminal 2. The doctor can also record an examination result and the like in the personal database 55 using the doctor terminal 2.

Furthermore, the doctor writes a question or an individual advice on the personal board in the individual advice module 54 using the doctor terminal 2. The individual advice module 54 records the written question or individual advice in the Q&A history database 59 while notifying the user terminal 1 by e-mail that the question or individual advice has been written. The user browses the question or individual advice written on the personal board in the individual advice module 54 using the user terminal 1.

The user writes a question or an answer to the question on the personal board in the individual advice module 54 using the user terminal 1.

The individual advice module 54 records the written question or the answer to the question in the Q&A history database 59 while notifying the doctor terminal 2 by e-mail that the question or answer has been written. The doctor browses the written question or answer on the personal board in the individual advice module 54 using the doctor terminal 2.

The individual advice module 54 reads the question, answer or individual advice recorded in the Q&A history database 59 to display it on the user terminal 1 or doctor terminal 2.

When the doctor judges that there is a problem with a individual set value based on the personal data, the doctor changes the personal set value recorded in the personal set value database 56 using the doctor terminal 2.

Further, if necessary, the doctor requests for the task of the individual advice to a psychological counselor or a dietitian from the doctor terminal 2 via the psychological counselor terminal 4 or dietitian terminal 3. In this case, using the psychological counselor terminal 4 or dietitian terminal 3, the psychological counselor or dietitian writes a question or an individual advice on the personal board in the individual advice module 54, and receives by e-mail a write-in notice of an answer or a question from the individual advice module 54.

At the completion of the individual advice work, the doctor transmits a finish notice to the advice selection module 51 using the doctor terminal 2.

In this way, the professionals have a hierarchical structure, in which the advice selection module 51 requests a certain type of professional (doctor, for example) for a judgment, and the professional delegates the judgment or the individual advice task to another type of professional (dietitian or psychological counselor, for example), if necessary.

The advice selection module 51 further has a function of automatically selecting a professional who makes a judgment based on the personal data with some problem. For example, in the case where the blood pressure exceeds a personal set value, the advice selection module 51 requests the doctor for a judgment; where there is a problem with diet, it requests the dietitian for a judgment; and where there is a mental problem, it requests the psychological counselor for a judgment.

In this way, switching between the automatic advice and the individual advice is performed.

Now refer to FIG. 4, generation of the automatic advice from the individual advice will be described.

If the user is in doubt about the automatic advice, the user writes a question on the personal board in the individual advice module 54 using the user terminal 1. The individual advice module 54 records the written question in the Q&A history database 59 while notifying the doctor terminal 2 by e-mail that the question has been written. The doctor browses the question written on the personal board in the individual advice module 54 using the doctor terminal 2.

The doctor then writes an individual advice on the personal board in the individual advice module 54 based on the personal data recorded in the personal database 55 using the doctor terminal 2. The individual advice module 54 records the written individual advice in the Q&A history database 59 while notifying the user terminal 1 by e-mail that the individual advice has been written. The user browses the individual advice written on the personal board in the individual advice module 54 using the user terminal 1.

Further, for an inquiry about the condition of the user, the doctor writes a question on the personal board in the individual advice module 54 using the doctor terminal 2. The individual advice module 54 records the written question in the Q&A history database 59 while notifying the user terminal 1 by e-mail that the question has been written. The user browses the question written on the personal board in the individual advice module 54 using the user terminal 1.

The user writes an answer to the question on the personal board in the individual advice module 54 using the user terminal 1. The individual advice module 54 records the written answer in the Q&A history database 59 while notifying the doctor terminal 2 by e-mail that the answer has been written. The doctor browses the answer written on the personal board in the individual advice module 54.

After that, the doctor writes an individual advice on the individual board in the individual advice module 54 using the doctor terminal 2, as described above. The questions and answers are repeated until an individual advice is presented by the doctor.

The process of transmission and reception of the questions, answers, and individual advice between the user and the doctor is recorded as a Q&A history in the Q&A history database 59.

The individual advice module 54 reads the questions, answers, and individual advices recorded in the Q&A history database 59 to display them on the user terminal 1 or doctor terminal 2.

When the presentation of the individual advice is completed, the advice construction module 53 extracts the Q&A history from the Q&A history database 59 to generate medical question sentences, conditional expressions, answers to medical questions, a medical question selecting condition, advice, and an advice algorithm based on the Q&A history.

Specifically, it is assumed that the medical question sentences or conditional expressions are Q0 to Qn; the answers to medical questions are R0 to Rn; and the medical question selecting condition is C. In this case, the medical question selecting condition C is decomposed into the medical question sentences or conditional expressions Q0 to Qn and the answers to medical questions R0 to Rn. Defining the advice as A, the advice algorithm can be expressed as follows:

If(Q0=R0 and Q1=R1 . . . Qn=Rn) then A

The advice construction module 53 sends an e-mail to the doctor terminal 2 notifying a confirmation request for the medical question sentences, conditional expressions, medical question selecting condition, advice, and advice algorithm.

Further, the advice construction module 53 allows the medical question sentences, conditional expressions, answers to medical questions, medical question selecting condition, advice and advice algorithm to be displayed on the doctor terminal 2. The doctor confirms the medical question sentences, conditional expressions, answers to medical questions, medical question selecting condition, advice, and advice algorithm displayed on the doctor terminal 2, and modifies them, if necessary. The doctor transmits the structures of modified medical sentences, conditional expressions, answers to medical questions, medical question selecting condition, advice, and advice algorithm from the doctor terminal 2 to the advice construction module 53.

The advice construction module 53 records the medical question sentences and answers to medical questions in the medical question database 58 while recording the conditional expressions, medical question selecting condition, advice, and advice algorithm in the algorithm database 57. The advice construction module 53 further ensures a storage area for the answers to medical questions in the personal database 55 while adding in the personal set value database 56 a storage area for personal set values for the advice algorithm.

In this way, the automatic advice is generated automatically from the individual advice.

Here, description will be made of an example of generation of the automatic advice from the individual advice. For example, the user writes a question on the personal board in the individual advice module 54 saying: “I have experienced my consciousness failing. What should I do?”

The doctor refers to the personal data in the user's personal database 55, and writes a question on the personal board in the individual advice module 54 saying: “Your blood glucose level is 38, but do you regularly take the hypoglycemic agent?”

The user writes an answer on the personal board in the individual advice module 54 saying: “I do three times a day. I take it each time even without a meal.”

The doctor writes an individual advice on the personal board in the individual advice module 54 saying: “That is a symptom of hypoglycemia. Take the medicine only before you have a meal.”

In this case, the advice construction module 53 displays the above Q&A history and an algorithm record form on the doctor terminal 2, and asks the doctor to input medical question sentences, conditional expressions, answers to medical questions, medical question selecting condition, advice, and advice algorithm, for example, in a following way:

Medical question sentence 1: “Do you ever experience your consciousness failing?”

Medical question sentence 2: “Do you take medicine also when you do not have a meal?”

Conditional expression 1: blood glucose level ≧average blood glucose level—100

Advice 1: “Since your blood glucose level is much lower than usual, it seems to be a hypoglycemia symptom. Take the medicine only before you have a meal.”

Advice algorithm:

if medical question sentence 1=Yes and medical question sentence 2=Yes and conditional expression 1=true then advice 1

where the answer to the medical question 1 is “Yes”, the answer to the medical question 2 is “Yes”, and the conditional expression 1 is satisfied, meaning that the advice 1 is to be presented.

Note that the advice construction module 53 may generate medical question sentences, conditional expressions, answers to medical questions, a medical question selecting condition, advice, and an advice algorithm based on the above Q&A history, and requests the doctor for confirmation.

Description will now be made of the operation of each of the components of the health care system according to the embodiment.

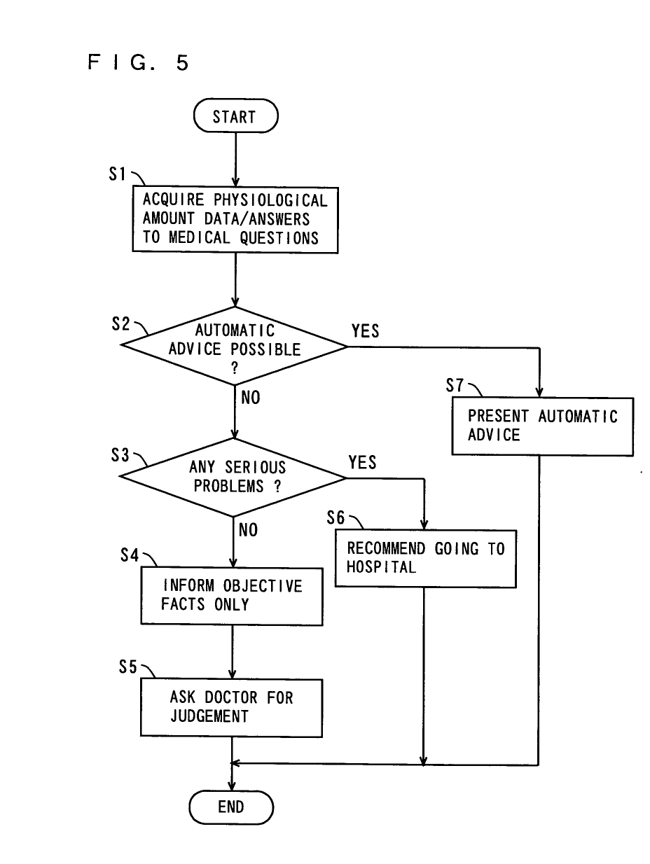

FIG. 5 is a flowchart showing the operation of the advice selection module 51 of FIG. 3 and FIG. 4. Here, referring to FIG. 5, a process of presenting an automatic advice by the advice selection module 51 is described.

First, the advice selection module 51 in the server 5 acquires the personal data from the personal database 55 (Step S1).

The advice selection module 51 then determines whether or not it is possible to present an automatic advice based on the personal data (Step S2). When determining that it is possible to present an automatic advice, the advice selection module 51 presents the automatic advice on the user terminal 1 (Step S7).

When determining that it is not possible to present an automatic advice, the advice selection module 51 determines whether or not the user has a serious health problem (Step S3). When determining that the user has a serious problem, the advice selection module 51 transmits a message for recommending the user to go to the hospital (Step S6), and informs that to the doctor via the doctor terminal 2.

When determining that the user has no serious problem, the advice selection module 51 transmits only the objective facts to the user terminal 1 (Step S4). Furthermore, the advice selection module 51 transmits a message to the doctor terminal 2 for requesting the doctor for a judgment (Step S5).

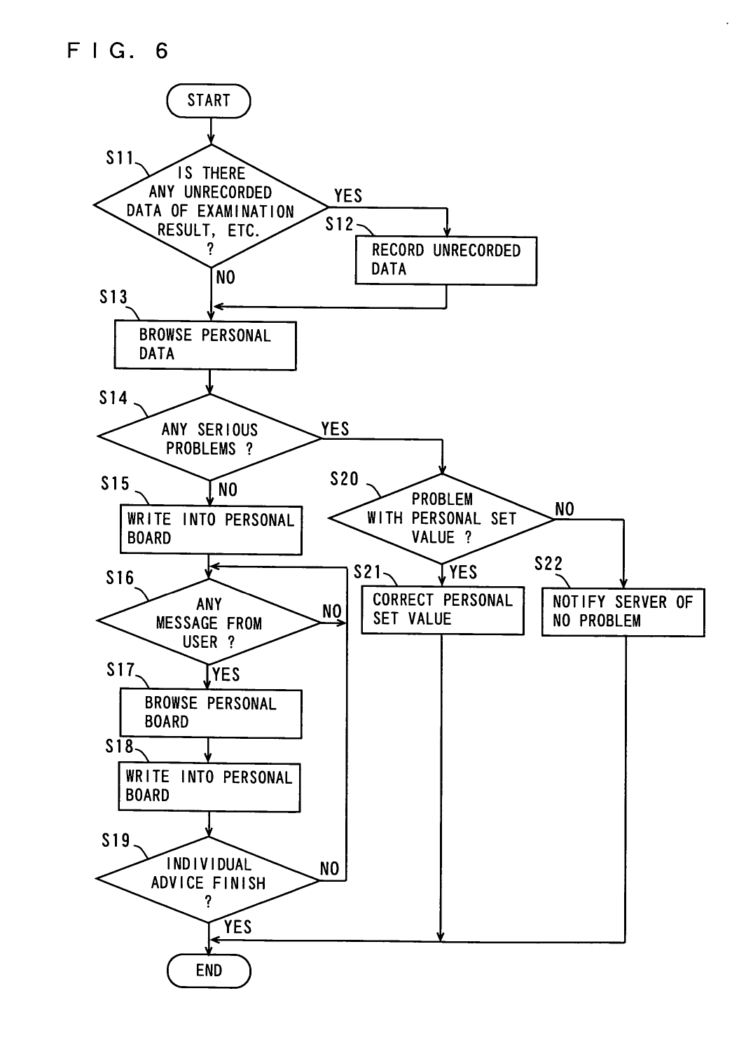

FIG. 6 is a flowchart showing the task of the doctor using the doctor terminal 2.

The doctor judges if there is any available data of user's examination result and the like which is not recorded in the personal database 55 (Step S1). When there is such unrecorded data, the doctor records it in the personal database 55 (Step S12).

The doctor browses the personal data displayed on the doctor terminal 2 (Step S13). The doctor then judges if the user has a serious health problem based on the personal data (Step S14).

When judging that the user has a serious problem, the doctor judges if there is any problem with personal set values recorded in the personal set value database 56 in the server 5 (Step S20). When judging that there is a problem with personal set values, the doctor corrects the personal set value recorded in the personal set value database 56 in the server 5, using the doctor terminal 2 (Step S21). When judging that there is no problem with personal set values, the doctor notifies the server 5 of no problem, using the doctor terminal 2 (Step S22).

When judging that there is no serious problem at Step S14, the doctor writes into the personal board in the individual advice module 54 in the server 5 (Step S15). Then, the doctor judges if there is a message written on the personal board from the user (Step S16). When there is a message written by the user, the doctor browses the personal board in the individual advice module 54 (Step S17), and writes into the personal board in the individual advice module 54 (Step S18). The doctor repeats the process of Steps S16 through S19 until the individual advice is completed. At the completion of the individual advice, the doctor completes the process.

For example, as personal set values for user's body temperature, it is assumed that a threshold value of presenting the automatic advice is a; a risk threshold value is b; and user's body temperature is t. Where t<a, an automatic advice is presented. Where a≦t<b, a professional is requested for a judgment. Where t≧b, a message is transmitted to the user for recommending him/her to go to the hospital or the doctor is notified.

When the professional judges that there is no problem with the personal set values, the advice selection module 51 is informed that there is no problem. When the professional judges that there is a problem with the personal set values, the risk threshold value b is modified to c. When the professional judges an individual advice to be necessary, he/she writes the individual advice on the personal board.

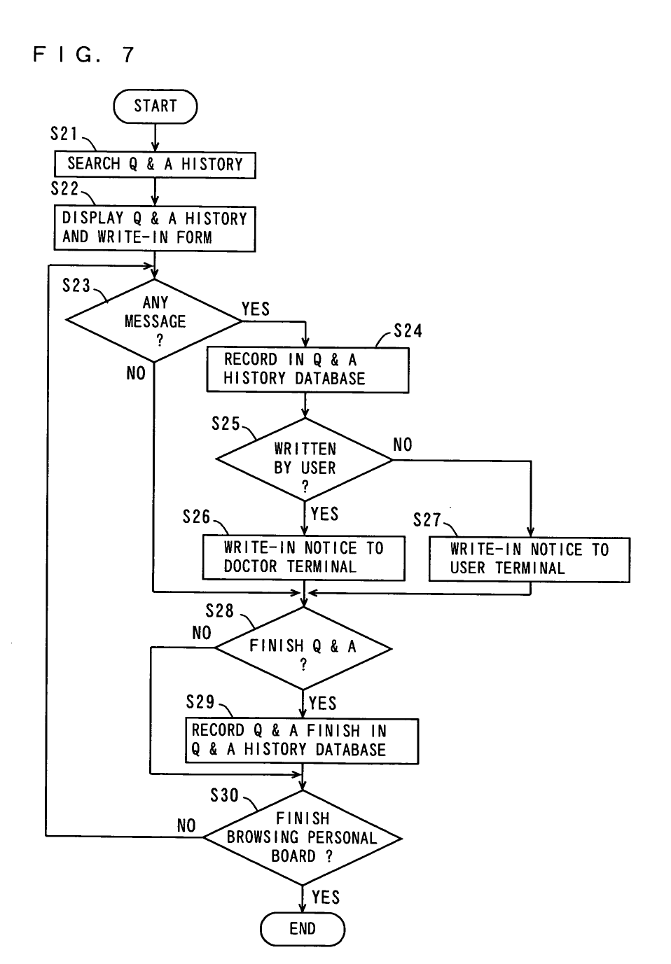

FIG. 7 is a flowchart showing the operation of the individual advice module 54 of FIG. 3 and FIG. 4. Here, referring to FIG. 7, description is made of the operation of the individual advice module 54 when the user or professional writes into and browses the personal board.

The individual advice module 54 first searches the Q&A history of the user which is recorded in the Q&A history database 59 (Step S21). Then, the individual advice module 54 allows the searched Q&A history and a write-in form to be displayed on the user terminal 1 or doctor terminal 2 (Step S22).

After that, the individual advice module 54 determines whether or not there is a message written on the personal board (Step S23). When there is a message written on the personal board, the individual advice module 54 records as a Q&A history the contents of the message in the Q&A history database 59 (Step S24).

Furthermore, the individual advice module 54 determines whether or not the message has been written by the user (Step S25). If so, the individual advice module 54 sends a write-in notice to the doctor terminal 2 by e-mail (Step S26). If not, the individual advice module 54 sends a write-in notice to the user terminal 1 by e-mail (Step S27).

The individual advice module 54 subsequently determines whether or not the Q&A (questions and answers) has been completed (Step S28). At the completion of the Q&A, the individual advice module 54 records the completion of the Q&A in the Q&A history database 59 (Step S29).

Note that when the Q&A has been completed by the user, the doctor terminal 2 is notified of the completion, and when the Q&A has been completed by the doctor, the user terminal 1 is notified of the completion.

The individual advice module 54 further determines whether or not the individual board browsing has been completed (Step S30). If not, it returns to Step S23 to repeat the process of Steps S23 through S30.

When the Q&A has not been completed at Step S28, the individual advice module 54 advances to Step S30. At the completion of the personal board browsing, individual advice module 54 completes the process.

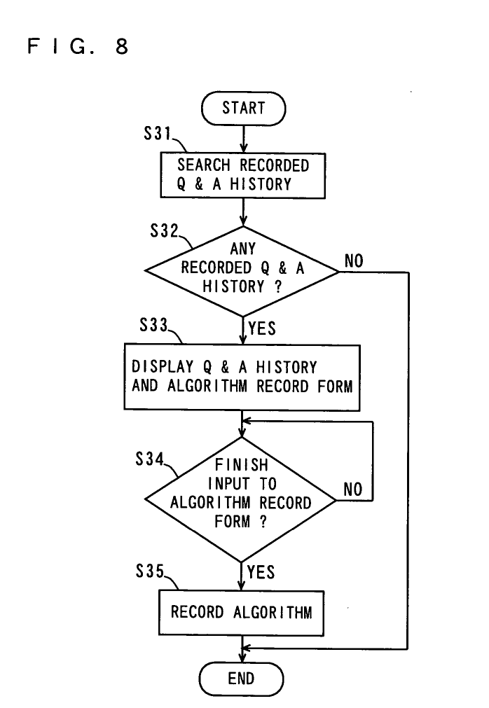

FIG. 8 is a flowchart showing the operation of the advice construction module 53 of FIG. 3 and FIG. 4. Here, referring to FIG. 8, description is made of the operation of the advice construction module 53 when the professional constructs an advice algorithm. Note that in the following description, a case in which a doctor as the professional constructs an advice algorithm will be discussed.

The advice construction module 53 first searches from the Q&A history database 59 the Q&A history which has already been recorded (Step S31). Then, the advice construction module 53 determines whether or not there is any recorded Q&A history in the Q&A history database 59 (Step S32).

When there is no recorded Q&A history, the advice construction module 53 completes the processing. When there is any recorded Q&A history, the advice construction module 53 allows the Q&A history and an algorithm record form to be displayed on the doctor terminal 2 (Step S33).

Then, the advice construction module 53 determines whether or not the doctor has completed an input to the algorithm record form (Step S34). At the completion of the entry on the algorithm record form, the advice construction module 53 records the algorithm (Step S35).

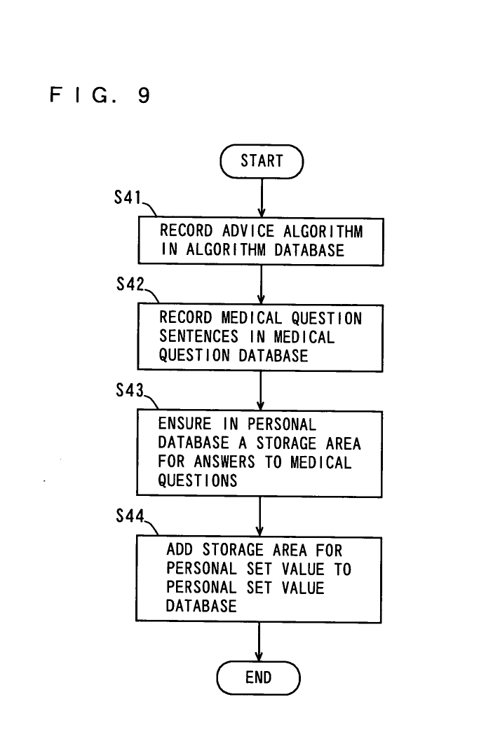

FIG. 9 is a flowchart showing a process of recording an algorithm in the operation of the advice construction module 53 of FIG. 3 and FIG. 4.

The advice construction module 53 records in the algorithm database 57 an algorithm including conditional expressions and a medical question selecting condition (Step S41).

The advice construction module 53 subsequently records in the medical question database 58 a medical question sentence supplied from the doctor terminal 2 (Step S42).

Further, the advice construction module 53 ensures a storage area for the answers to the medical question recorded at Step S42 in the personal database 55 (Step S43). The advice construction module 53 also adds in the personal set value database 56 a storage area for a personal set value (Step S44).

As described in the foregoing, in the health case system according to the embodiment, at normal times the automatic advice is presented automatically to the user based on the personal data. Therefore, the user can readily receive advice. When it is not possible to present the automatic advice, the individual advice based on the judgment of a professional is presented to the user. Therefore, when it is difficult to make a judgment, the user can readily receive appropriate individual advice from the professional.

In this case, the professional is requested for a judgment only when it is not possible to present an automatic advice, and therefore, the workload of the professional does not increase.

It is accordingly possible to efficiently present appropriate advice according to the health condition of the user.

Further, the automatic advice is generated automatically from the individual advice based on the messages written on the personal board in the individual advice module 54. This allows acquisition of professional know-how without requiring much effort, leading to further enhanced performance of the function of presenting the automatic advice.

Note that the user terminal 1 may comprise in part or all of the functions of the server 5 according to the embodiment. In the case where the user terminal 1 comprises all the functions of server 5, the automatic advice presenting function and individual advice presenting function can be implemented by connecting only the user terminal 1 to the network 6 without the use of the server 5.

In the embodiment, the user terminal 1 corresponds to a user terminal; the doctor terminal 2, dietitian terminal 3, psychological counselor terminal 4 correspond to professional terminals; the server 5 corresponds to a host computer; the network 6 corresponds to a communication line. And, the personal database 55 corresponds to a data storage device; the algorithm database 57 corresponds to an advice storage device; the advice selection module 51 corresponds to a first advice presenting unit; the individual advice module 54 corresponds to a second advice presenting unit. In addition, the advice construction module 53 corresponds to an advice construction unit; the personal board corresponds to an information storage device; the personal set value database 56 corresponds to a criterion value storage device. Furthermore, the medical question presenting module 52 corresponds to a medical question presenting device. A first advice corresponds to the automatic advice; a second advice corresponds to the individual advice; and a criterion value corresponds to the personal set value.

Note that each of the first advice presenting unit, second advice presenting unit, advice construction unit, and medical question presenting unit may be composed of a computer such as a CPU and a program, or may be composed of a hardware such as an electronic circuit.

Although the present invention has been described and illustrated in detail, it is clearly understood that the same is by way of illustration and example only and is not to be taken by way of limitation, the spirit and scope of the present invention being limited only by the terms of the appended claims.

What is claimed is:

1. A health care system comprising:
a user terminal which is used by a user;
a professional terminal which is used by a professional; and
a host computer, wherein
said user terminal, said professional terminal, and said host computer are interconnected via a communication line, and said host computer includes:
a data storage device that stores data relating to the health condition of the user supplied by said user terminal;
an advice storage device that stores a plurality of advices relating to the health condition as first advices;
a first advice presenting unit that determines whether or not a first advice can be presented based on the data stored in said data storage device, and when a first advice can be presented, selects any of the plurality of first advices stored in said advice storage device to present the advice on said user terminal, while requesting said professional terminal for a judgment when said first advice cannot be presented; and
a second advice presenting unit that presents on said user terminal advice from said professional terminal as a second advice.

2. The health care system according to claim 1, wherein
said host computer constructs a first advice based on the second advice presented on said user terminal by said second advice presenting unit, and further comprises an advice construction unit that makes said advice storage device store the constructed first advice.

3. The health care system according to claim 2, wherein
said second advice presenting unit includes an information storage device which can be written to and read from by said user terminal and said professional terminal, and
said advice construction unit constructs said first advice based on the information written to said information storage device.

4. The health care system according to claim 1, wherein
said host computer further comprises a criterion value storage device that stores a criterion value set for each user for presenting advice, and
said first advice presenting unit determines whether or not a first advice can be presented and selects a first advice, based on the data stored in the data storage device and the criterion value stored in the criterion value storage device.

5. The health care system according to claim 4, wherein
the criterion value stored in said criterion value storage device can be set and changed by said professional terminal.

6. The health care system according to claim 4, wherein
when a first advice cannot be presented, said first advice presenting unit determines whether or not there is a serious problem based on the criterion value stored in said criterion value storage device.

7. The health care system according to claim 1, wherein
said professional terminal includes a plurality of terminals having a hierarchical structure, and
when a first advice cannot be presented, said first advice presenting unit requests a judgment to a highest level terminal of the plurality of terminals having the hierarchical structure.

8. The health care system according to claim 1, wherein
said professional terminal includes a plurality of terminals, and
when a first advice cannot be presented, said first advice presenting unit selects any of said plurality of terminals and requests the selected terminal for a judgment, based on the data stored in said data storage device.

9. The health care system according to claim 1, wherein
said host computer further comprises a medical question presenting unit that presents a medical question on said user terminal,
said data storage device stores as said data user's physiological amount data supplied by said user terminal and an answer supplied by said user terminal in response to the medical question by said medical question presenting unit, and
said first advice presenting unit determines whether or not a first advice can be presented and selects said first advice, based on the physiological amount data and the answer stored in said data storage device.

10. The health care system according to claim 1, wherein
when the data falls short to determine if said first advice can be presented and select said first advice, said first advice presenting unit acquires sufficient data from the medical question performed by said medical question presenting unit.

11. A health care apparatus comprising:
a data storage device that stores data relating to the health condition of a user supplied by the user;
an advice storage device that stores a plurality of advices relating to the health condition as first advices;
a first advice presenting unit that determines whether or not a first advice can be presented based on the data stored in said data storage device, and when a first advice can be presented, selects any of the plurality of first advices stored in said advice storage device to present the advice to the user, while requesting a professional for a judgment when said first advice cannot be presented; and
a second advice presenting unit that presents advice from the professional to the user as a second advice.

12. The health care apparatus according to claim 11, further comprising an advice construction unit that constructs a first advice based on the second advice presented to the user by said second advice presenting unit, and makes said advice storage device store the constructed first advice.

13. The health care apparatus according to claim 12, wherein
said second advice presenting unit includes an information storage device which can be written to and read from by the user and the professional, and
said advice construction unit constructs said first advice based on the information written to said information storage device.

14. The health care apparatus according to claim 11, further comprising a medical question presenting unit that presents a medical question to the user, wherein
said data storage device stores as said data user's physiological amount data supplied by the user and an answer supplied by the user in response to the medical question by said medical question presenting unit, and
said first advice presenting unit determines whether or not a first advice can be presented and selects said first advice, based on the physiological amount data and the answer stored in said data storage device.

15. The health care apparatus according to claim 11, wherein
when the data falls short to determine if said first advice can be presented and select said first advice, said first advice presenting unit acquires sufficient data from the medical question performed by said medical question presenting unit.

16. A health care method comprising the steps of:
storing data relating to the health condition of a user supplied by the user;
storing a plurality of advices relating to the health condition as first advices;
determining if a first advice can be presented based on said stored data, and when a first advice can be presented, selecting any of said stored plurality of first advices to present the advice to the user, while requesting a professional for a judgment when said first advice cannot be presented; and
presenting advice from the professional to the user as a second advice.

17. The health care method according to claim 16, further comprising the step of constructing a first advice based on said second advice presented to the user, and storing the constructed first advice.

18. The health care method according to claim 17, wherein
said step of constructing advice includes constructing said first advice based on information stored in an information storage device which can be written to and read from by the user and the professional.

19. The health care method according to claim 16, further comprising the step of presenting a medical question to the user, wherein
said step of storing data includes storing as said data user's physiological amount data supplied by the user and an answer supplied by the user in response to said medical question, and
said step of presenting said first advice includes determining if a first advice can be presented and selecting said first advice, based on said stored physiological amount data and answer.

20. The health care method according to claim 16, wherein
said step of presenting said first advice includes acquiring sufficient data from said medical question, when data falls short to determine if said first advice can be presented and select said first advice.

21. A computer-readable health care program which allows said computer to implement the processes of:
storing data relating to the health condition of a user supplied by the user;
storing a plurality of advices relating to the health condition as first advices;
determining if a first advice can be presented based on said stored data, and when a first advice can be presented, selecting any of said stored plurality of first advices to present the advice to the user, while requesting a professional for a judgment when said first advice cannot be presented; and
presenting advice from the professional as a second advice to the user.
